(19) United States
(12) Patent Application Publication
(10) Pub. No.: US 2022/0054892 A1
(43) Pub. Date: Feb. 24, 2022

(54) System and Method for Providing Real-Time Feedback Related to Fitness Training

(71) Applicants: Craig North, Sunnyvale, CA (US); Sapna Todwal, San Jose, CA (US)

(72) Inventors: Craig North, Sunnyvale, CA (US); Sapna Todwal, San Jose, CA (US)

(21) Appl. No.: 16/999,251

(22) Filed: Aug. 21, 2020

(51) Int. Cl. A63B 24/00; G06F 3/048; A61B 5/11; G06F 3/01; A61B 5/00

(52) U.S. Cl. CPC A63B 24/0062; A63B 2024/0068; A61B 5/1118; G06F 3/011; A61B 5/744; A61B 5/7405; A61B 5/0015; A61B 5/0004; A63B 24/0087; A63B 24/0075; A61B 2562/0219; A61B 2562/0223; A61B 2560/0223; A63B 2024/0096; G06F 3/048

(57) ABSTRACT

A system and method including the steps of analyzing an expert training video; extracting reference data from the expert training video; storing the extracted reference data to a master database; loading a selected profile from the master database; communicating with a sensor implement placed on a predetermined landmark of a user's body; loading a selected workout, exercise or activity to perform after the sensor implement is calibrated; retrieving a reference data for the selected workout, exercise or activity, wherein said reference data comprises a pose estimation from the expert training video; recording user's movement based on data received from the sensor implement; rendering the user's movements on the GUI screen; comparing values collected from the sensor recording step with the retrieved reference data; and providing a user feedback based on the comparing step.

### CROSS-REFERENCE TO RELATED APPLICATIONS

Not applicable.

### RELATED CO-PENDING U.S. PATENT APPLICATIONS

Not applicable.

### INCORPORATION BY REFERENCE OF SEQUENCE LISTING PROVIDED AS A TEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to fitness applications. More particularly, certain embodiments of the invention relate to real-time fitness tracking applications.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The incorporation of fitness into the average person's daily life may greatly improve overall health and mental well-being. In modern society, it has become more and more difficult to incorporate a healthy fitness regimen into a busy day to day schedule. The additional time required to commute to and from a gym as well as complete a workout can be daunting to the average person. Further, gym memberships are often expensive and are not financially feasible for many users, and even if a person applies for a membership, new members must often overcome social anxiety and body confidence issues often associated with poor physical health. It may be difficult to choose a new fitness regimen, and in many cases prior knowledge or expertise is required to safely and adequately perform a new routine.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that technology based fitness applications may provide an alternative means for effectively and inexpensively fitting a fitness routine into an everyday schedule. Conventionally, home fitness regimens require a user to follow along with a work out being performed on a screen. This provides the convenience of exercising at home without the need to go to a gym, but often does not provide any personalization that may be beneficial to the average user. Further, it is difficult to determine if a user's fitness is improving over time, or if a user is performing the exercises correctly. Technology based approaches often incorporate sensors for more personalized tracking of exercises and fitness level, but may be overly expensive and typically provide feedback to the user after the user completes an exercise routine.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates an exemplary system for providing real-time virtual fitness services which may include, but not limited to, exercises, fitness training, and/or performances, in accordance with an embodiment of the present invention;

FIGS. 2A-2B illustrate exemplary pose estimation of expert training videos wherein FIG. 2A depicts an expert training video and FIG. 2B depicts the expert training video with pose estimation, in accordance with an embodiment of the present invention;

FIG. 3 illustrates exemplary sensor information, in accordance with an embodiment of the present invention;

FIG. 4 illustrates a block diagram depicting an exemplary back end flow of data, in accordance with an embodiment of the present invention;

FIG. 5 illustrates a block diagram depicting exemplary sensor data processing, in accordance with an embodiment of the present invention;

FIG. 6 is a flow chart illustrating an exemplary process for an initial setup of virtual fitness services, in accordance with an embodiment of the present invention;

FIG. 7 is a flow chart illustrating an exemplary process for using virtual fitness services, in accordance with an embodiment of the present invention;

FIG. 8 illustrates an exemplary user interface for providing user information to real-time virtual fitness services, in accordance with an embodiment of the present invention;

FIGS. 9A-9C illustrates user interfaces for real-time virtual fitness services, wherein FIG. 9A illustrates an exemplary activity selection user interface, FIG. 9B illustrates an exemplary activity preview user interface, and FIG. 9C illustrates an exemplary activity user interface, in accordance with an embodiment of the present invention;

FIG. 10 is a block diagram depicting an exemplary client/server system which may be used by an exemplary web-enabled/networked embodiment of providing real-time feedback, but not limited to, exercises, fitness training, and/or performances of the present invention; and

FIG. 11 illustrates a block diagram depicting an exemplary client/server communication system, which may be used by an exemplary web-enabled/networked embodiment of providing real-time feedback, but not limited to, exercises, fitness training, and/or performances of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hollinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

While embodiments herein may be discussed in terms of a processor having a certain number of bit instructions/data, those skilled in the art will know others that may be suitable such as 16 bits, 32 bits, 64 bits, 128s or 256 bit processors or processing, which can usually alternatively be used. Where a specified logical sense is used, the opposite logical sense is also intended to be encompassed.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the web sites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, FireFox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

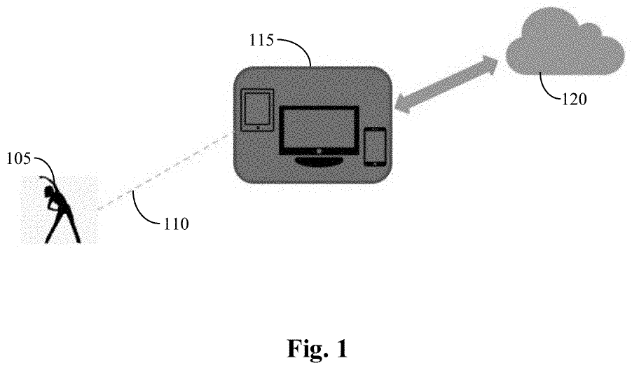

FIG. 1 illustrates an exemplary system for providing real-time virtual fitness services which may include, but not limited to, exercises, fitness training, and/or performances, in accordance with an embodiment of the present invention. Sensor implements 105 may be placed on a user on various places on the user's body, such as, but not limited to, wrists, feet, hips/waist, back, shoulders, knees, elbows, ankles, between shoulder plates, etc. depending on the activity to be performed. Sensors 105 may be wearable sensors placed on a user by any means known in the art, such as, but not limited to, straps, adhesive, as part of a body suit, as pocket(s) in custom long-sleeved shirts (e.g. between the shoulder plates and wrists), etc. It should be appreciated by one skilled in the art that singular or multiple sensor implements 105 may be used and the locations of sensors 105 are not limited to the examples listed above. Sensors 105 may comprise internal measurement units (IMU), but any other means of body tracking may be used, such as, but not limited to, electromagnetic tracking systems, acoustic tracking systems, optical tracking devices, mechanical tracking systems, etc. In one embodiment, sensors 105 may be able to track various types of data, such as, but not limited to, positional data, velocity, angular rate, etc. using a combination of wearable accelerometers, gyroscopes, magnetometers, etc. Additionally, at least one of sensors 105 may be equipped to track health information, such as, but not limited to, heart rate, oxygen levels, body temperature, body impedance measurements, etc. via for example, but not limited to, heart rate monitors, oximeters, thermometers, electrodes, etc. One or more sensors 105 may communicate with singular, plural, or combination of device(s) 115 via Bluetooth Low Energy (BLE) 110. In an embodiment, sensors 105 may comprise a communication interface adapted to communicate with device(s) 115 via communication channel 110, as will be appreciated by one skilled in the art. Sensors 105 may communicate with device 115 via any means, such as, but not limited to, wired or wireless communication, infrared communication, local area network (LAN), Wireless Fidelity (WiFi), Bluetooth (BLE), near field communication (NFC), Zigbee, etc. Further, device 115 may be singular or multiple devices, and may include, for example, but not limited to, smart phones, tablets, personal computers, smart televisions, or any other type of device with computing capabilities known in the art. Device 115 may be able to communicate with cloud services 120 through network interface 145 to store data to be used with the application including training module 140, such as, but not limited to, user data, reference data, etc. In one embodiment, cloud services 120 may be adapted to communicate with device(s) 115 via communication channel 125, but not limited to, wired communication, wireless communication, infrared communication, local area network (LAN), WAN, Wireless Fidelity (WiFi), etc. Additionally, device 115 may store data locally in physical storage/memory 135, such as, but not limited to, solid state drives (SSD), hard drives, non-volatile random access memory (NVRAM), flash memory, RAM, ROM, etc. Device(s) 115 may display training and/performance feedback on a graphical user interface 130.

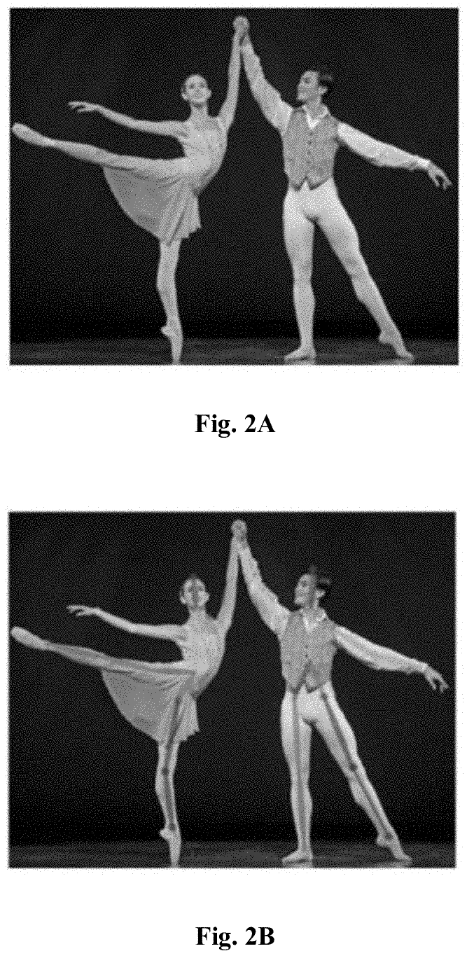

FIGS. 2A-2B illustrate exemplary pose estimation of expert training videos wherein FIG. 2A depicts an expert training video and FIG. 2B depicts the expert training video with pose estimation, in accordance with an embodiment of the present invention. With reference to FIG. 2A, expert training videos may be analyzed to procure reference data to be stored in a main/master database. The reference data may be later used while a user is performing an activity as a baseline for virtual feedback to be provided to the user, as will be described in greater detail below. Referring now to FIG. 2B, expert training videos may be analyzed to procure, for example, without limitation, pose estimation, along other types of reference data. Additionally, an expert may be equipped with sensors to procure expert sensor data during the filming of an expert training video. The expert sensor data may be used in tandem with reference data from an analyzed expert training data to produce more accurate reference data to store on a main database.

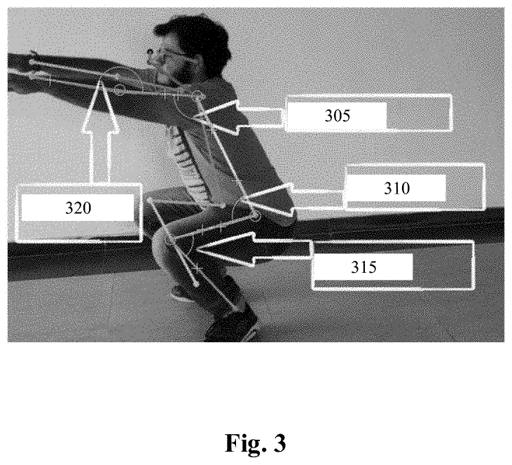

FIG. 3 illustrates exemplary sensor information, in accordance with an embodiment of the present invention. After the sensors 105 are placed on a user, sensor data may be collected and used for pose estimation. Analysis of body landmarks such as, but not limited to, wrists, shoulders, hips/waist, knees, elbows, ankles, between shoulder plates and landmarks tracking may be used for pose estimation and/or exercise classification. Accelerometer/Gyroscope sensors 105 with Bluetooth low energy communication and a specialized app is used to record user movements which produces accelerometer/gyroscopic data on shoulders 305, hips/waist 310, knees 315, and elbows 320 to be used for pose estimation and/or exercise classification. This is achieved by run time comparison between the stored meta-data for the video which may include the body landmark positions and the current positions as received from sensors 105 and providing the appropriate feedback depending on the activity. Once the exercise is classified, expert training videos may be further analyzed in exercise counting and used to compile sensor compatible data with training module 140. The above data may be used to create feedback messages for different exercises and stored in a master database. As will be appreciated by one skilled in the art, various body landmarks may be measured, and are not limited to those described above. These exemplary body landmarks may be compared with reference data associated with expert training videos, and may be used to create feedback for the user.

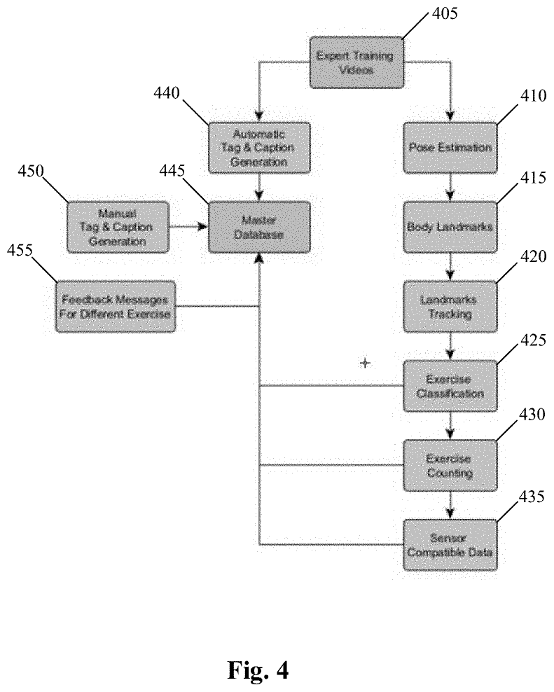

FIG. 4 illustrates a block diagram depicting an exemplary back end flow of data, in accordance with an embodiment of the present invention. Expert training videos 405 may be analyzed to gain reference data to be stored in master database 445. In one embodiment, analysis may be achieved either by using existing tools that can derive various body joint locations and then mapping it to the respective sensor values, or by having an expert follow the same video by wearing sensors 105 on one or more body locations like the wrists, shoulders, hips/waist, knees, elbows, ankles, between shoulder plates and then training module 140 noting these positions for the different key moments in the video. Pose estimation 410 may include the data amplitude and frequency of each of the body landmarks 415. Analysis of body landmarks 415 and landmarks tracking 420 may be used for exercise classification 425. Once the exercise is classified, expert training videos 405 may be further analyzed in exercise counting 430 and used to compile sensor compatible data 435. The above data may be used to create feedback messages for different exercise 455, and stored in master database 445. Additionally, captions and tags may be generated for each expert training video. Expert training videos may be automatically analyzed 440 and/or manually analyzed 450 to generate tags and captions associated with expert training videos 405 and stored in master database 445.

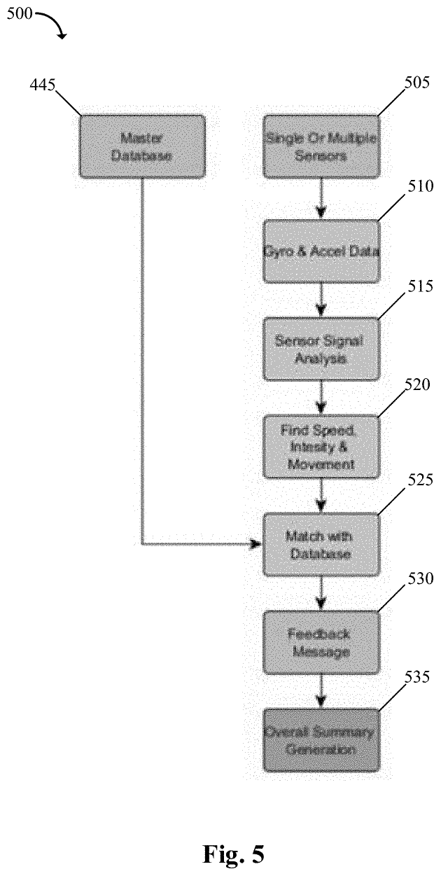

FIG. 5 illustrates a block diagram depicting exemplary sensor data processing, in accordance with an embodiment of the present invention. With reference to both FIG. 4 and FIG. 5, single or multiple sensors 105 may generate data to be compared with data from master database 445 to generate training feedback for the user with a Graphical User Interface (GUI). Gyroscopic and acceleration data 510 may be provided by single or multiple sensors 105 while a user is performing an activity. Gyroscopic and acceleration data may be transmitted to an application and the application may perform sensor signal analysis 515 to find speed, intensity, and movement 520 of the user. Once analyzed, the data may be matched with database 525, comparing the user's data with reference data compiled in master database 445. Subsequently, feedback message 530 may be generated and displayed to the user in real-time. After the activity is complete, overall summary generation 535 may be performed, and the overall summary may be displayed to the user. In some embodiments, accelerometer/gyroscope sensors 105 including Bluetooth low energy communication with a specialized application may be used to record user movements and use it for fitness training. This is achieved by run time or real time comparison between the stored meta-data of the expert training video which may include the stored body landmark positions and the current positions as received from sensors 105.

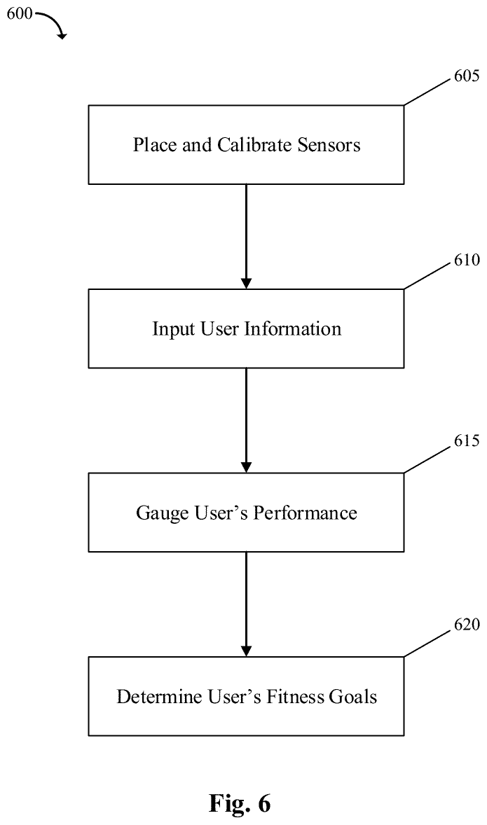

FIG. 6 is a flow chart illustrating an exemplary process for an initial setup of virtual fitness services, in accordance with an embodiment of the present invention. An initial setup of virtual fitness services process 600 begins with a step 605 where a user may place sensors 105 on the user's body and go through a process for calibrating the sensors. The sensor placement may depend on the activity to be performed, and sensors 105 may be placed on, for example, without limitation, wrists, feet, waist, back, etc. Once sensors are in place, the application may guide the user to calibrate sensors in a first time set up by performing exercises such as, but not limited to, waving each hand, moving each foot, performing simple exercises, etc. in order to locate the sensors, determine if the sensors were placed properly, and, if not, instruct the user to reconfigure sensor placement. Process 600 then continues to a step 610 where the application prompts the user to input user information. User information may include basic information such as, without limitation, name, email, date of birth, gender, etc., medical information, such as, without limitation, family medical history, weight, height, blood pressure, etc., as well as any other information that may be needed by the application. Process 600 may then gauge a user's initial performance in a step 615. Sensor data may be used to determine a user's resting heart rate and oxygen levels to serve as a baseline for future activities. Further, sensor data may be used to determine various body dimensions such as, but not limited to, the distance from the top of the head to the neck, etc. The application may instruct the user to perform basic exercises, such as, but not limited to, pushups, running in place, various stretches, etc. to gauge the user's initial fitness level, range of motion, and flexibility, and may store the initial fitness information as part of the user's profile. The process may then continue to a step 620 in which the application determines the user's fitness goals. The user may manually enter their fitness goals or choose from a list of options provided by the application. The list of options may include, for example, without limitation, weight loss, increase stamina, increase flexibility, gain muscle, etc. Further, the user may input specific fitness goals such as, but not limited to, target weight, target mile time, number of exercises per week, amount of time to exercise per week, etc. After the user's fitness goals are determined, initial set up is complete.

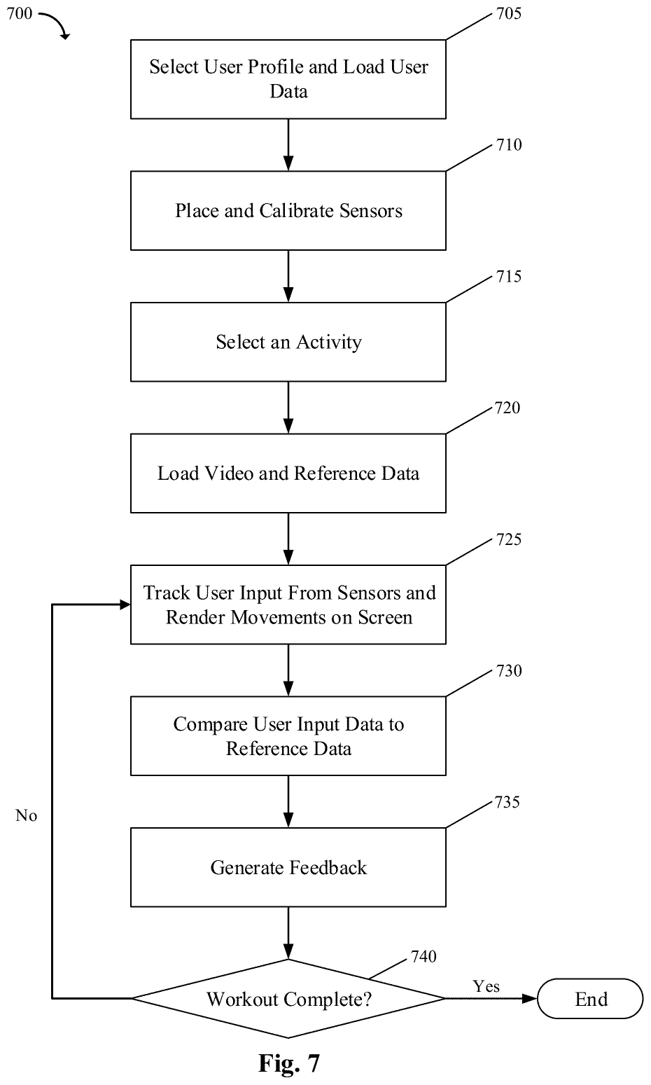

FIG. 7 is a flow chart illustrating an exemplary process for using virtual fitness services, in accordance with an embodiment of the present invention. A process 700 may begin with a step 705 where the user is prompted to select a user profile and the application loads the user's data. User data may either be stored locally or via cloud services of Global Network 120, or a combination of both. Once user data is successfully loaded, the user may place and calibrate sensors 105 in a step 710. User data related to the placement of sensors 105 may be used to facilitate step 710 based on the first time set up previously performed by the user. Process 700 continues with a step 715 where the user is prompted to select an activity to perform. The user may manually search for an activity, browse by category, or select from a list of suggested activities. For example, without limitation, the application may suggest activities based on different criteria such as recently performed exercise, based on fitness goals, related activities, etc. After an activity is selected, process 700 continues to a step 720 where the application loads selected video and reference data based on different criteria such as recently performed exercise, fitness goals, related activities. The application also loads the meta-data for the video which may include the reference data for expected sensor values during important key moments in the video, and also the expected feedback during key moments, if there is a deviation. Video and reference data may be retrieved from cloud services 120 of the global network, or may optionally be loaded from local storage if the user downloaded the data for offline use. When the video starts playing, the application may start tracking user movements by using motion sensor inputs from wearable sensors that the user is wearing via BLE. The application also loads the meta-data for the video which primarily includes the reference data for expected sensor values during important key moments in the video, and also the expected feedback during those key moments, if there is a deviation. The application continues to track user input from the sensors and render the user's movements on the GUI screen in a step 725. User's input may be tracked in real-time, and a virtual representation of the user may be displayed on the GUI screen based on the data transmitted by the sensors. Process 700 may then continue to a step 730 where user input data is compared to reference data and the application generates feedback in a step 735. The values collected from tracking the user movements are then constantly compared with the reference values and based on that the appropriate feedback is selected. The feedback can then be displayed, but not limited to, on the screen in words, shown in the form of correction using an on-screen avatar for user, and/or provided as audio feedback. In another embodiment, the feedback may be displayed on the GUI screen and/or the application may provide audio feedback to accompany the video. For example, without limitation, the application may prompt the user to correct their form, slow down, speed up, etc. Process 700 then determines if the workout is complete in a step 740. If not, process 700 proceeds back to step 725 and the activity continues. If so, process 700 terminates.

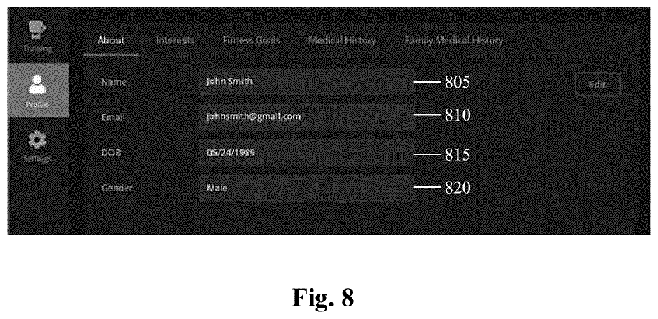

FIG. 8 illustrates an exemplary user interface for providing user information to real-time virtual fitness services, in accordance with an embodiment of the present invention. Various user information may be displayed while the user access the user's profile, and may be edited at any time. For example, without limitation, the user's basic information is displayed in FIG. 8. Basic information may include, without limitation, name 805, email 810, date of birth (DOB) 815, gender 820, etc.

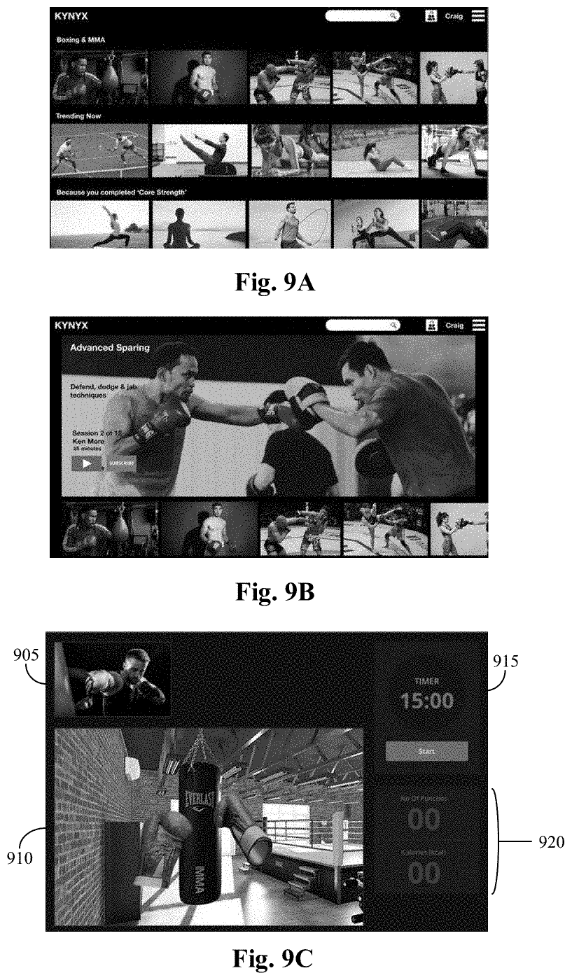

FIGS. 9A-9C illustrates user interfaces for real-time virtual fitness services, wherein FIG. 9A illustrates an exemplary activity selection user interface, FIG. 9B illustrates an exemplary activity preview user interface, and FIG. 9C illustrates an exemplary activity user interface, in accordance with an embodiment of the present invention. With reference to FIG. 9A, the application may display a series of different activities for the user to perform. The application may suggest activities based on relevant categories, such as, but not limited to, boxing and mixed martial arts (MMA), based on popularity, based on previously completed activities, etc. With reference to FIG. 9B, once an activity is selected, a preview for the activity may be displayed to the user. The preview may include information such as, but not limited to, a title, duration, number of sessions, description, etc. With reference to FIG. 9C, once a user continues to the workout, the user may be presented with a user interface related to the workout. Instructional video 905 may be used to demonstrate the activity to the user. The application may also use sensor data to generate a user rendering 910. A timer 915 may be displayed, indicating the remaining duration of the activity. Finally, workout information 910 may be displayed to the user to keep track of user progress. Workout information may be specific to the activity performed, such as, but not limited to, number of punches, or based on sensor data, such as, but not limited to, calories burned, heart rate, oxygen level, etc.

Those skilled in the art will readily recognize, in light of and in accordance with the teachings of the present invention, that any of the foregoing steps and/or system modules may be suitably replaced, reordered, removed and additional steps and/or system modules may be inserted depending upon the needs of the particular application, and that the systems of the foregoing embodiments may be implemented using any of a wide variety of suitable processes and system modules, and is not limited to any particular computer hardware, software, middleware, firmware, microcode and the like. For any method steps described in the present application that can be carried out on a computing machine, a typical computer system can, when appropriately configured or designed, serve as a computer system in which those aspects of the invention may be embodied. Such computers referenced and/or described in this disclosure may be any kind of computer, either general purpose, or some specific purpose computer such as, but not limited to, a workstation, a mainframe, GPU, ASIC, etc. The programs may be written in C, or Java, Brew or any other suitable programming language. The programs may be resident on a storage medium, e.g., magnetic or optical, e.g., without limitation, the computer hard drive, a removable disk or media such as, without limitation, a memory stick or SD media, or other removable medium. The programs may also be run over a network, for example, with a server or other machine sending signals to the local machine, which allows the local machine to carry out the operations described herein.

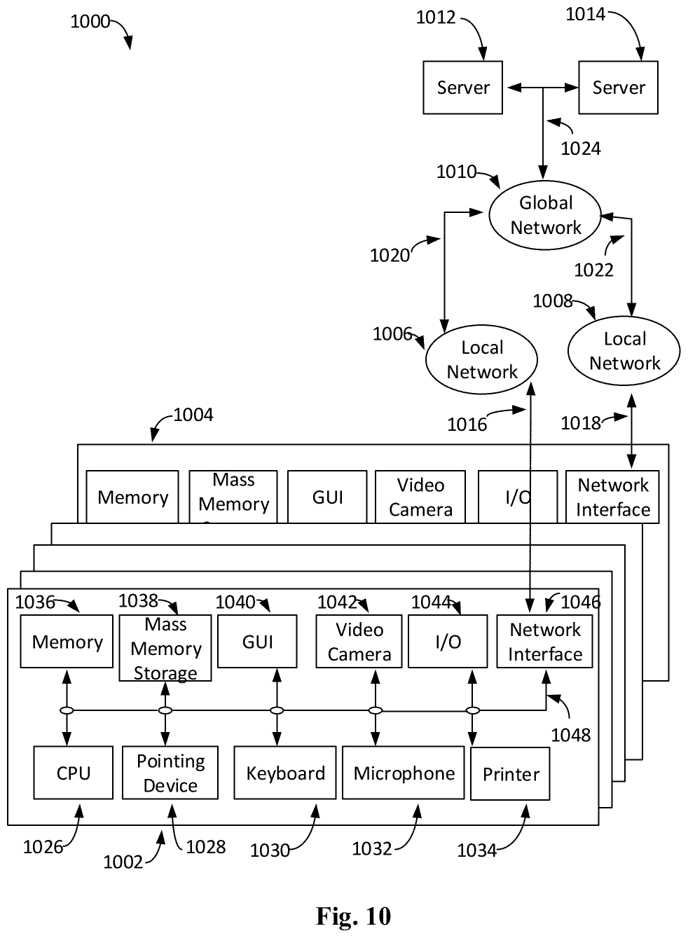

FIG. 10 is a block diagram depicting an exemplary client/server system which may be used by an exemplary web-enabled/networked embodiment of providing real-time feedback, but not limited to, exercises, fitness training, and/or performances of the present invention.

A communication system 1000 may include a multiplicity of devices/clients with a sampling of clients denoted as a device 115 and a client 1004 for communicating with singular or multiple sensors 105 (i.e. 105a, 105b, 105c), a multiplicity of local networks with a sampling of networks denoted as a local network 1006 and a local network 1008, a global network/cloud services 120, a database, and a server denoted as a server 1012 and a master database445.

Device 115 may communicate bi-directionally with local network 1006 via a communication channel 125. Client 1004 may communicate bi-directionally with local network 1008 via a communication channel 1018. Local network 1006 may communicate bi-directionally with global network 120 for providing cloud services via a communication channel 1020. Local network 1008 may communicate bi-directionally with global network 120 via a communication channel 1022. Global network 120 may communicate bi-directionally with server 1012 and master database 445 via a communication channel 1024. Server 1012 and master database 445 may communicate bi-directionally with each other via communication channel 1024. Furthermore, device 115, 1004, local networks 1006, 1008, global network 120 , servers 1012, and master database 445 may each communicate bi-directionally with each other. Device 115 may communicate bi-directionally with sensors 105 via Bluetooth, wired communication, infrared communication, local area network (LAN), Wireless Fidelity (WiFi), near field communication (NFC), Zigbee, etc. With reference to both FIG. 4 and FIG. 5, single or multiple sensors 105 may generate data to be compared with data from master database 445 to generate training feedback for the user through Graphical User Interface (GUI) 130. Gyroscopic and acceleration data 510 may be provided by single or multiple sensors 105 while a user is performing an activity.

In one embodiment, global network 120 may operate as the Internet providing cloud services. It will be understood by those skilled in the art that communication system 1000 may take many different forms. Non-limiting examples of forms for communication system 1000 include local area networks (LANs), wide area networks (WANs), wired telephone networks, wireless networks, or any other network supporting data communication between respective entities.

Devices 115 and 1004 may take many different forms. Non-limiting examples of devices 115 and 1004 include personal computers, personal digital assistants (PDAs), cellular phones, smartphones, tablets, personal computers, smart televisions, or any other type of device with computing capabilities known in the art.

Device 115 may include a CPU 1026, a pointing device 1028, a keyboard 1030, a microphone 1032, a printer 1034, a local memoryl35, a master database 445, a Graphical User Interface (GUI)130, a video camera 1042, an input/output interface 1044 and a network interface125.

CPU 1026, pointing device 1028, keyboard 1030, microphone 1032, printer 1034, memory 135, master database 445, GUI130, video camera 1042, input/output interface 1044 and network interface 125 may communicate in a unidirectional manner or a bi-directional manner with each other via a communication channel 1048. Communication channel 1048 may be configured as a single communication channel or a multiplicity of communication channels.

CPU 1026 may be comprised of a single processor or multiple processors. CPU 1026 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general purpose microprocessors.

As is well known in the art, local memory 135 is used typically to transfer application data and instructions to CPU 1026 in a bi-directional manner. Memory 135, as discussed previously, may include any suitable computer-readable media, intended for data storage, such as those described above excluding any wired or wireless transmissions unless specifically noted. Master database 445 may also be coupled bi-directionally to CPU 1026 and provides additional data storage capacity and may include any of the computer-readable media described above. Master database 445 may be used to store expert training videos 405, expert training video reference data such as, but not limited to, meta-data like pose estimation, data amplitude & frequency of each body landmarks, type of exercise, and exercise count, feedback messages or training feedback for different exercise programs 455, applications or programs, sensor data, user information and the like and is typically a secondary storage medium such as a solid state drives (SSD), non-volatile random access memory (NVRAM), a hard disk, etc. It will be appreciated that the information retained within master database 445, may, in appropriate cases, be incorporated in standard fashion as part of memory 135 as virtual memory.

CPU 1026 may be coupled to GUI130. GUI 130 enables a user to view training feedback and an overall summary of an activity. The user may be shown a list of fitness activities and prompted to select an activity to perform. Various user information may be displayed while the user access the user's profile, and may be edited at any time. For example, without limitation, the user's basic information is displayed in FIG. 8. Basic information may include, without limitation, name 805, email 810, date of birth (DOB) 815, gender 820, etc. CPU 1026 may be coupled to pointing device 1028. Non-limiting examples of pointing device 1028 include computer mouse, trackball and touchpad. Pointing device 1028 enables a user with the capability to maneuver a computer cursor about the viewing area of GUI 130 and select areas or features in the viewing area of GUI130. CPU 1026 may be coupled to keyboard 1030. Keyboard 1030 enables a user with the capability to input alphanumeric textual information to CPU 1026 such as a user information which may include basic information such as, without limitation, name, email, date of birth, gender, etc., medical information, such as, without limitation, family medical history, weight, height, blood pressure, etc., as well as any other information that may be needed by the application. The user may manually enter their fitness goals or choose from a list of options provided by the application. The list of options may include, for example, without limitation, weight loss, increase stamina, increase flexibility, gain muscle, etc. Further, the user may input specific fitness goals such as, but not limited to, target weight, target mile time, number of exercises per week, amount of time to exercise per week, etc. The user may select a user profile and the application loads the user's data. CPU 1026 may be coupled to microphone 1032. Microphone 1032 enables audio produced by a user to be recorded, processed and communicated by CPU 1026. CPU 1026 may be connected to printer 1034. Printer 1034 enables a user with the capability to print information to a sheet of paper. CPU 1026 may be connected to video camera 1042. Video camera 1042 enables video produced or captured by user to be recorded, processed and communicated by CPU 1026.

CPU 1026 may also be coupled to input/output interface 1044 that connects to one or more input/output devices such as such as CD-ROM, video monitors, track balls, mice, keyboards, microphones, touch-sensitive displays, transducer card readers, magnetic or paper tape readers, tablets, styluses, voice or handwriting recognizers, or other well-known input devices such as, of course, other computers.

Finally, CPU 1026 optionally may be coupled to network interface 145 which enables communication with an external device such as a database or a computer or telecommunications or internet network using an external connection shown generally as communication channel 125, which may be implemented as a hardwired or wireless communications link using suitable conventional technologies. CPU 1026 may be coupled to network interface 145 which enables communication with an external device such as device 105 using an external connection shown generally as communication channel 110, which may be implemented as, but not limited to, wired or wireless communication, infrared communication, local area network (LAN), Wireless Fidelity (WiFi), Bluetooth (BLE), near field communication (NFC), Zigbee, etc. With such a connection, CPU 1026 might receive information from wearable devices, or might output information to GUI 130 in the course of performing the method steps described in the teachings of the present invention.

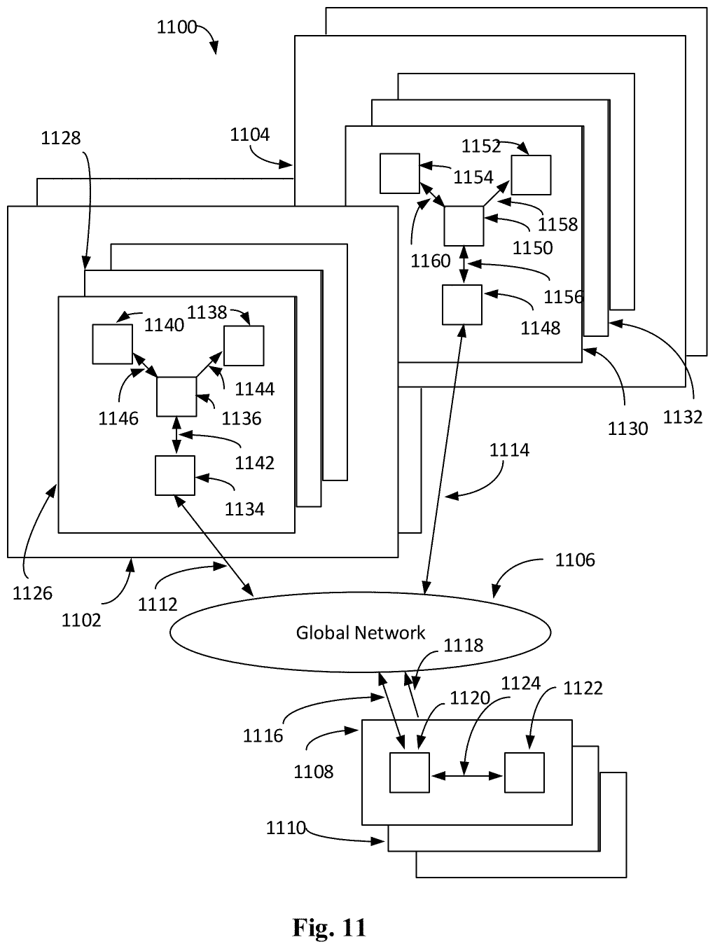

FIG. 11 illustrates a block diagram depicting an exemplary client/server communication system, which may be used by an exemplary web-enabled/networked embodiment of providing real-time feedback, but not limited to, exercises, fitness training, and/or performances of the present invention.

A communication system 1100 includes a multiplicity of networked regions with a sampling of regions denoted as a network region 1102 and a network region 1104, a global network120, and a sampling of a database and a server denoted as a master database 445 and a server device 1108.

Network region 1102 and network region 1104 may operate to represent a network contained within a geographical area or region. Non-limiting examples of representations for the geographical areas for the networked regions may include postal zip codes, telephone area codes, states, counties, cities and countries. Elements within network region 1102 and 1104 may operate to communicate with external elements within other networked regions or within elements contained within the same network region.

In some implementations, global network 120 may operate as the Internet and provides cloud services to the system. It will be understood by those skilled in the art that communication system 1100 may take many different forms. Non-limiting examples of forms for communication system 1100 include local area networks (LANs), wide area networks (WANs), Wireless Fidelity (WiFi), near field communication (NFC), Zigbee, wired telephone networks, cellular telephone networks or any other network supporting data communication between respective entities via hardwired or wireless communication networks. Global network 120 may operate to transfer information between the various networked elements and provide cloud services.

Server device 1108 and master database 445 may operate to execute software instructions, store information, support master database 445 operations and communicate with other networked elements. Non-limiting examples of software and scripting languages which may be executed on server device 1108 and master database 445 include C, C++, C# and Java.

Network region 1102 may operate to communicate bi-directionally with global network 120 via a communication channel 125. Network region 1104 may operate to communicate bi-directionally with global network 120 via a communication channel 125. Server device 1108 may operate to communicate bi-directionally with global network 120 via a communication channel 125. Master database 445 may operate to communicate bi-directionally with global network 120 via a communication channel 125. Network region 1102 and 1104, global network 120, server devices 1108 and master database 445 may operate to communicate with each other and with every other networked device located within communication system 1100.

Server device 1108 includes a networking device 1120 and a server 1122. Networking device 1120 may operate to communicate bi-directionally with global network 120 via communication channel 125 and with server 1122 via a communication channel 1124. Server 1122 may operate to execute application/software instructions and store information such as training videos, meta-data for the video, accelerometer/gyroscopic sensor data, training feedback, etc. to master database 445.

Network region 1102 includes a multiplicity of clients with a sampling denoted as a device 115 and a client 1128. Device 115 includes a networking device 1134, a processor 1136, a GUI 130 and an interface device 1140. Non-limiting examples of devices for GUI 130 include monitors, televisions, cellular telephones, smartphones and PDAs (Personal Digital Assistants). Non-limiting examples of interface device 1140 include pointing device, mouse, trackball, scanner and printer. Networking device 1134 may communicate bi-directionally with global network 120 via communication channel 125 and with processor 1136 via a communication channel 1142. Graphical User Interface (GUI) 130 may receive information from processor 1136 via a communication channel 1144 for presentation to a user for viewing such as but not limited to instructions for the user to perform basic exercises, a virtual representation of the user, a feedback, etc. For example, without limitation, the information may prompt the user to correct their form, slow down, speed up, etc. Interface device 1140 may operate to send control information to processor 1136 and to receive information from processor 1136 via a communication channel 1146. Network region 1104 includes a multiplicity of clients with a sampling denoted as a device 115 and a client 1132. Device 115 includes a networking device 1148, a processor 1150, a GUI 130 and an interface device 1154. Non-limiting examples of devices for Graphical User Interface (GUI) 130 include monitors, televisions, cellular telephones, smartphones and PDAs (Personal Digital Assistants). Non-limiting examples of interface device 1140 include pointing devices, mousse, trackballs, scanners and printers. Networking device 1148 may communicate bi-directionally with global network 120 via communication channel 1114 and with processor 1150 via a communication channel 1156. GUI 130 may receive information from processor 1150 such as but not limited to feedback messages for different exercise 455, feedback message 530, overall summary, user's basic information is displayed in FIG. 8 which may include, without limitation, name 805, email 810, date of birth (DOB) 815, gender 820, etc. via a communication channel 1158 for presentation to a user for viewing. Interface device 1154 may operate to send control information to processor 1150 and to receive information from processor 1150 via a communication channel 1160.

For example, consider the case where a user interfacing with device 115 may want to execute a networked application. A user may enter the user profile for the networked application using interface device 1140. The user profile may be communicated to processor 1136 via communication channel 1146. Processor 1136 may then communicate the user profile to networking device 1134 via communication channel 1142. Networking device 1134 may then communicate the user profile to global network 120 via communication channel 1112. Global network 120 may then communicate the user profile to networking device 1120 of server device 1108 via communication channel 1116. Networking device 1120 may then communicate the user profile to server 1122 via communication channel 1124. Server 1122 may receive the user profile and after processing the user profile may communicate return information to networking device 1120 via communication channel 1124. Networking device 1120 may communicate the return information to global network 120 via communication channel 1116. Global network 120 may communicate the return information to networking device 1134 via communication channel 1112. Networking device 1134 may communicate the return information to processor 1136 via communication channel 1142. Processor 1136 may communicate the return information to GUI 1118 via communication channel 1144. User may then view the return information on GUI 130.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC § 112 (6)/(f) it is intended that the corresponding means for and/or steps for carrying out the claimed function are the ones that are locally implemented within the jurisdiction of the USA, while the remaining aspect(s) performed or located remotely outside the USA are not intended to be construed under 35 USC § 112 (6) pre-AIA or 35 USC § 112 (f) post MA. In some embodiments, the methods and/or system components which may be located and/or performed remotely include, without limitation:

It is noted that according to USA law, all claims must be set forth as a coherent, cooperating set of limitations that work in functional combination to achieve a useful result as a whole. Accordingly, for any claim having functional limitations interpreted under 35 USC § 112 (6)/(f) where the embodiment in question is implemented as a client-server system with a remote server located outside of the USA, each such recited function is intended to mean the function of combining, in a logical manner, the information of that claim limitation with at least one other limitation of the claim. For example, in client-server systems where certain information claimed under 35 USC § 112 (6)/(f) is/(are) dependent on one or more remote servers located outside the USA, it is intended that each such recited function under 35 USC §112 (6)/(f) is to be interpreted as the function of the local system receiving the remotely generated information required by a locally implemented claim limitation, wherein the structures and or steps which enable, and breath life into the expression of such functions claimed under 35 USC § 112 (6)/(f) are the corresponding steps and/or means located within the jurisdiction of the USA that receive and deliver that information to the client (e.g., without limitation, client-side processing and transmission networks in the USA). When this application is prosecuted or patented under a jurisdiction other than the USA, then “USA” in the foregoing should be replaced with the pertinent country or countries or legal organization(s) having enforceable patent infringement jurisdiction over the present patent application, and “35 USC § 112 (6)/(f)” should be replaced with the closest corresponding statute in the patent laws of such pertinent country or countries or legal organization(s).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112(6) (post AIA 112(f)) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing real-time fitness tracking applications according to the present invention will be apparent to those skilled in the art. Various aspects of the invention have been described above by way of illustration, and the specific embodiments disclosed are not intended to limit the invention to the particular forms disclosed. The particular implementation of the real-time fitness tracking applications may vary depending upon the particular context or application. By way of example, and not limitation, the real-time fitness tracking applications described in the foregoing were principally directed to real-time virtual activity feedback implementations; however, similar techniques may instead be applied to live-streamed personal trainer group lessons and soft skills training where for example, the user can be trained on how to have the right body language during a presentation, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre AIA) or 35 USC 112(f) post-AIA. Otherwise, no limitations from the specification are to be read into any claims, unless those limitations are expressly included in the claims.

What is claimed is:

1. A method comprising the steps of:
executing on a device with a CPU, digital instructions stored on a computer-readable media, wherein the device comprises at least one of, a smart phone, a tablet, a personal computer, and a smart television;
executing an application software that is configured to provide real-time virtual fitness service;
analyzing an expert training video;
extracting reference data from the expert training video;
storing the extracted reference data to a master database;
loading a selected profile from the master database, wherein the selected profile comprises at least one of name, email, date of birth, gender, and medical information;
communicating with a sensor implement placed on a predetermined landmark of a user's body;
loading a selected workout, exercise or activity to perform after the sensor implement is calibrated;
retrieving a reference data for the selected workout, exercise or activity, wherein said reference data comprises a pose estimation from the expert training video;
recording user's movement based on data received from the sensor implement;
rendering the user's movements on the GUI screen;
comparing values collected from the sensor recording step with the retrieved reference data; and
providing a user feedback based on the comparing step.

2. The method of claim 1, in which said sensor implement comprises at least one of a Gyroscope, an Accelerometer, a magnetometer, temperature reader, oximeter and a heart rate monitor.

3. The method of claim 2, in which said feedback comprises a prompt for the user to correct form, slow down, Of speed up, improve activity, improve ability, improve strength , or improve cardiovascular health.

4. The method of claim 3, in which said feedback prompt comprises an on-screen avatar and audio feedback.

5. The method of claim 4, further comprising the steps of tracking the sensor implement, wherein the sensor implement is configured to be operable for providing at least one of, a positional data, a velocity, and an angular rate of the landmark.

6. The method of claim 5, further comprising the steps of displaying user's movements on the GUI screen.

7. The method of claim 5, further comprising the steps of:
determining if the workout, exercise or activity being performed is completed; and
terminating the application software based on the determining step.

8. The method of claim 7, further comprising the steps of:
displaying on a GUI coupled to the device CPU, a prompt to select a user profile from a list of user profiles; and
sensing on the GUI, the selection of a user profile from the list of user profiles.

9. The method of claim 8, further comprising the steps of:
ascertaining if the sensor implement was properly placed on the predetermined landmark of a user's body;
displaying an instruction to reconfigure the sensor placement based on the ascertaining step that the sensor implement was not placed properly; and
calibrating the sensor implement based on the ascertaining step that the sensor implement was placed properly.

10. A method comprising:
steps of executing on a device with a CPU, digital instructions stored on a computer-readable media, wherein the device comprises at least one of, a smart phone, a tablet, a personal computer, and a smart television;
steps of executing an application software that is configured to provide real-time virtual fitness services;
steps of analyzing an expert training video, extracting reference data from the expert training video, and storing the reference data to a master database;
steps of loading a selected profile from the master database, wherein the selected profile comprises at least one of name, email, date of birth, gender, and medical information;
steps of communicating with a plurality of sensors placed on predetermined landmarks of a user's body;
steps of loading a selected workout, exercise or activity to perform after the plurality of sensors is calibrated;
steps of retrieving a reference data for the selected workout, exercise or activity, wherein said reference data comprises a pose estimation from an expert training video;
steps of recording user's movement based on data received from the plurality of sensors;
steps of rendering the user's movements on the GUI screen;
steps of comparing values collected from the plurality of sensors recording step with the retrieved reference data; and
steps of providing a user feedback based on the comparing step.

11. The method of claim 10, in which said feedback comprises a prompt for the user to correct form, slow down, speed up, improve activity, improve ability, improve strength , or improve cardiovascular health.

12. The method of claim 11, in which said feedback prompt comprises an on-screen avatar and audio feedback.

13. The method of claim 12, in which said plurality of sensors comprises at least one of a plurality of Gyroscopes, a plurality of Accelerometers, a plurality of magnetometers, a temperature reader, oximeter and a heart rate monitor.

14. The method of claim 13, further comprising the steps of tracking the plurality of sensors, wherein each of the plurality of sensors is configured to be operable for providing at least one of, a positional data, a velocity, and an angular rate of the landmark.

15. The method of claim 14, further comprising the steps of displaying user's movements on the GUI screen.

16. The method of claim 15, further comprising the steps of:
determining if the workout, exercise or activity being performed is completed; and
terminating the application software based on the determining step.

17. The method of claim 16, further comprising the steps of:
displaying on a GUI coupled to the device CPU, a prompt to select a user profile from a list of user profiles; and
sensing on the GUI, the selection of a user profile from the list of user profiles.

18. The method of claim 10, further comprising the steps of:
ascertaining if each of the plurality of sensors was properly placed on the predetermined landmark of a user's body;
displaying an instruction to reconfigure a misplace sensor based on the ascertaining step that the sensor was not placed properly; and
calibrating the plurality of sensors based on the ascertaining step that each of the plurality sensor was placed properly.

19. The method of claim 18, wherein said communication step is performed using wireless communication.

20. A method comprising:
steps of executing on a device with a CPU, digital instructions stored on a computer-readable media;
steps of executing on the device an application software that is configured to provide real-time virtual fitness services, in which the device including at least one of, a smart phone, a tablet, a personal computer, and a smart television;
steps of displaying on a GUI coupled to the device CPU, a prompt to select a user profile from a list of user profiles;
steps of sensing on the GUI, the selection of a user profile from the list of user profiles;
steps of loading the selected profile from a master database, wherein the selected profile comprises at least one of name, email, date of birth, gender, and medical information;
steps of communicating wirelessly with at least one sensor placed on a predetermined landmark of a user's body;
steps of ascertaining if the at least one sensor was properly placed on the predetermined landmark of a user's body;
steps of displaying an instruction to reconfigure the sensor placement based on the ascertaining step that the sensor was not placed properly;
steps of calibrating the sensor based on the ascertaining step that the sensor was placed properly;
steps of displaying a workout, exercise or activity selection list to perform;
steps of retrieving a reference data of the workout, exercise or activity selected, said reference data comprises a pose estimation from an expert training video;
steps of tracking the sensor;
steps of recording user's movement based on data received from the sensor;
steps of rendering user's movements on the GUI screen;
steps of comparing values collected from said tracking step with the reference data; and
steps of providing a feedback based on the comparing step, wherein the feedback comprises a prompt for the user to correct form, slow down, or speed up.
